(19) United States
(12) Patent Application Publication
(10) Pub. No.: US 2007/0087036 A1
(43) Pub. Date: Apr. 19, 2007

(54) Edible film for transmucosal delivery of nutritional supplements

(76) Inventors: Maurice E. Durschlag, Charlotte, NC (US); Gary S. Kehoe, Glendale, AZ (US)

Correspondence Address: PERKINS COIE LLP, POST OFFICE BOX 1208, SEATTLE, WA, 98111-1208, US

(21) Appl. No.: 11/417,676

(22) Filed: May 3, 2006

Related U.S. Application Data
(60) Provisional application No. 60/677,679, filed on May 3, 2005.
(60) Provisional application No. 60/677,717, filed on May 4, 2005.

(51) Int. Cl. A61K 47/00

(52) U.S. Cl. 424/439

(57) ABSTRACT

In one embodiment of the present invention a composition is provided comprising a film layer wherein the film layer rapidly dissolves in an oral cavity and a coating comprising a powder matrix, wherein the coating is applied to at least one side of the film layer and wherein the powder matrix comprises a nutritional supplement, an adhesive, a bulking agent, a flow agent, and a sweetener.

### CROSS-REFERENCE TO RELATED APPLICATIONS

The present application claims priority to: (a) U.S. Provisional Patent Application No. 60/677,679, filed May 3, 2005 (Atty Dkt No. 57778.8005.US00), (b) U.S. Provisional Patent Application No. 60/677,717, filed May 4, 2005 (Atty Dkt No. 57778.8005.US01), (c) U.S. patent application Ser. No. 10/713,544, filed Nov. 14, 2003 (Atty Dkt No. 57778.8001.US01), which claims priority to U.S. Provisional Patent Application No. 60/426,598, filed Nov. 14, 2002 (Atty Dkt No. 57778.8001.US00), and U.S. Provisional Patent Application No. 60/497,186 filed Aug. 22, 2003 (Atty Dkt No. 57778.8003.US00), (d) U.S. patent application Ser. No. 10/402,273, filed Mar. 28, 2003 (Atty Dkt No. 57778.8002.US00), (e) U.S. patent application Ser. No. 10/921,770, filed Aug. 18, 2004 (Atty Dkt No. 57778.8003.US01), which claims priority to U.S. Provisional Patent Application No. 60/497,186, filed Aug. 22, 2003 (Atty Dkt No. 57778.8003.US00), and (f) U.S. patent application Ser. No. 10/706,810, filed Nov. 12, 2003 (Atty Dkt No. 57778.8004.US00), which claims priority to U.S. Provisional Patent Application No. 60/426,598, filed Nov. 14, 2002 (Atty Dkt No. 57778.8001.US00), the disclosures of all of which are incorporated by reference herein in their entirety, including drawings.

### FIELD OF THE INVENTION

The present invention relates to the use of edible films for transmucosal delivery of nutritional supplements.

### BACKGROUND OF THE INVENTION

There is a long history of human consumption of vitamins, minerals, herbs, and other nutritional substances. The health food industry might be considered to have emerged in the 1800s with Reverend Sylvester Graham's invention of the Graham cracker and Kellogg's development of packaged cereals.

By the middle of the 20th Century consumers were encouraged to consume three nutritional meals a day and to avoid foods with negative effects on the body. Emphasis in the latter part of the Century shifted from avoidance of food with negative effects on the body to monitoring the quantities consumed. It was at this time that advances in science and medicine accelerated the understanding and popularity of functional food groups.

The health food industry grew at a 15 percent compounded growth rate from 1992 to 1998 driven primarily by the demographics of the baby bowmen and the population's interest in a health-conscious lifestyle. The growth rate slowed substantially during 1999 and is expected to be around 10 percent for the next three years. Various sources estimate 1999 sales of domestic natural products at between $25 billion and $35 billion.

Over the past several years, the increased popularity of alternative medicine and the growing number of health conscious consumers have contributed to increased sales of nutritional supplements. This trend is expected to continue, with sales increasing further in the future. The public awareness of the positive effects of vitamins and nutritional supplements on health has been heightened by widely publicized reports of scientific findings supporting such claims. The non-elasticity of demand for natural food products has also underwritten the growth of the industry. We observe that demand for health food products appears to be less price-sensitive than demand for regular foods. We believe Consumers are willing to pay premium prices for such products fin two important reasons:

1. They believe in the health benefits of consuming such products

2. Their busy lifestyles demand the convenience of vitamins and nutritional supplements

Increasing numbers of health professionals recognize the benefits of nutritional supplements and advocate their use in preventing illnesses such as heart disease and strokes. Similarly, governments and health care providers looking to cut healthcare costs emphasize preventative healthcare. Recent studies indicate a correlation bet the regular consumption of selected nutritional supplements and reduced incidences of a wide range of conditions such as cancer, heart disease, stroke, and arthritis.

The aging of the U.S. population and a corresponding shift toward focus on preventative health measures, prominently including good diet, will continue to increase demand for vitamins and nutritional supplements. According to the U.S. Census Bureau, the 36-and-older age group of consumers, which represents a substantial majority of regular users of vitamins and nutritional supplements, is expected to grow significantly faster through year 2010 than the general population. Industry sources also report that vitamin consumers are taking more vitamins and nutritional supplements per day than in the past. Consolidation, strong demographic trends, and more science-based nutrition is expected in the coming years.

Because of the foregoing reasons there is a desire in the field for alternate methods to deliver nutritional suipplements.

### SUMMARY OF THE INVENTION

The present invention relates to various oral/buccal transmucosal systems for delivering nutritional supplements to mammal and/or human bodies.

Such oral/buccal transmucosal systems include quick dissolve strips, thin-film composites, powders, gels, sprays, time release lozenge or reservoir packets, and other oral/buccal transmucosal drug/substance delivery systems.

One such oral/buccal transmucosal system that could be used to deliver nutritional supplements is the 3M™ Cydot™ System offered in several configurations including matrix and reservoir designs. Another such oral/buccal transmucosal system that could be used to deliver nutritional supplements is Zengen Inc.'s “oral strip bilayer system” which is being used in Chloraseptic Relief Strips™. Yet another system could be a “tea bag” device similar to a Skoal Bandit™ product.

Such nutritional supplements include but are not limited to Iron, Sodium, Calcium, Magnesium, Carbohydrates, Protiens, Sugars (Glucose), Zinc, Molybdenum, Copper, Potassium, Manganese, Chlorides, Bicarbonate and Carbonate, Aluminium, Arsenic, Bromine, Cadmium, Chromium, Chlorine, Cobalt, Fluorine, Iodine, Manganese, Molybdenum Nickel, Phosphorus, Selenium, Silicon, Vanadium, Zinc, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantethine, Folic Acid, Vitamin B12, “Unofficial” B Vitamins including Choline and Inositol, Vitamin P (bioflavonoids), and/or other vital nutrients, in addition to various homeopathic/alternative substances.

### DETAILED DESCRIPTION OF THE PREFERRED EMBODIMENTS

In order to fully understand the manner in which the above-recited details and other advantages and objects according to the invention are obtained, a more detailed description of the invention will be rendered by reference to specific embodiments thereof.

Therapeutics can be effectively delivered across the mucous membrane. Transmucosal delivery is particularly attractive because these membranes are very thin and permeable. Such properties allow for the rapid uptake of a drug (substance) into the body. This efficient uptake allows drug (substances) to bypass some of the body's natural defenses and enhances the effect of the therapeutic. Transmucosal delivery systems offer several benefits over other methods of delivery including:

Direct Absorption: Absorption through the mucous membrane leads directly to the circulatory system. This allows drugs (substances) to bypass the gastrointestinal tract as well as first pass liver metabolism. This is important for biological therapeutics.

Rapid Onset: Drugs (substances) directly enter the circulatory system, which allows the therapeutic to be rapidly transported to the site of need. The faster the drug/substance reached its target area, the faster it can begin to elicit its desired effect.

Lower Dosage: The avoidance of the gastrointestinal tract and first pass metabolism means that much less of the drug can be administered to achieve the same effect, allowing for lower dosages to be administered and fewer side effects.

Transmucosal drug delivery is generally classified into three systems:

Nasal Transmucosal—Products in this category include nasal sprays, pumps, and gels. The majority of the drugs delivered to the nasal passage are anti-inflammatories.

Oral/Buccal Transmucosals—These systems make use of saliva to release the therapeutic. Products include mucoadhesives, quick-dissolve strips or other quick dissolve delivery systems, reservoir pouches or packets, and solid lozenge formulations.

Vaginal or Urethral Suppositories—Delivery systems in this category are designated to be absorbed directly by the vaginal or penile capillary beds.

A viscous polysaccbaride matrix designed to trap foreign particles that may enter the system coats the mouth, nasal passage, vagina and urethra. This is a defense, which prevents damage to delicate tissues and capillary beds which lie directly underneath the epithelium. Though the mucous membrane protects the body from foreign matter and pathogens, the area is much more permeable than mucous membrane. This permeability allows drugs delivered to the mucous membrane to quickly enter into circulation.

Nutritional solutions may be used to orally replace nutrients lost during vomiting, diarrhea, heavy perspiration, other forms of fluid loss, and/or other natural nutrient deficiencies related the body's genetic makeup or current genetic state, in addition to other causes of nutrient deficiency.

Transmucosal Nutrient Supplements may be used in mild, moderate, and/or sever cases of nutrient loss.

Transmucosal delivery of nutrient supplements offer advantages over oral delivery when negative issues relating to the gastrointestinal tract, the stomach, substance digestion and absorption, swallowing, protocol compliance, and substance effectiveness as well as other issues with respect to gastrointestinal metabolism are considered.

An appropriate edible film carrier for use with embodiments of the present invention can be selected by one of ordinary skill in the art depending upon factors including the desired rate of dissolution, desired oral feel for the user, the compatibility of the thin film carrier and the active ingredients, production constraints, costs, or other factors. The film can also be thick or thin depending upon these same factors.

The desired rate for dissolution can vary depending of the specific application for the edible film. For example, for immediate delivery of the active ingredient, the film can be manufactured to rapidly dissolve in the oral cavity thus delivering the entire dosage of active ingredient at one time. The film can also be manufactured to dissolve over an extended period regulating the amount of active material delivered to the oral cavity over a desired length of time.

Specific film formulations and methods of manufacture are known in the art, for example see U.S. Pat. No. 5,948,430 to ZERBE et al., incorporated herein by reference. Each film formulation usually comprises film formers, bulking agents, softeners, intense artificial sweeteners, sugar alcohol, natural sweeteners, flavors, cooling agents, surfactants, coloring agents, oils, and drying agents. These ingredients are well known and widely available in the food industry.

The primary ingredient for an edible film according to the present invention is the film former, which in most cases can be any water soluble film former. Film formers include but are not limited to pullulan, guar gum, pectin, xanthan gum, alginates, gelatin, starches (including corn, potato, rice or tapioca), modified starches, matltodextrins, wheat gluten, carboxymethylcellulose, carrageenan konjac or locust bean gum.

An example of edible film according to the present invention is described comprising a bi-layer film. The film consists of one water soluble layer that serves as a substrate layer or active layer and a second dry coat layer. The second dry coat layer settles into the substrate layer affixing itself to that bottom layer. While active ingredients may be contained in either layer, preferably the second dry coat layer will contain one or more active ingredients such as menthol or benzocaine or both. The dry coat layer is applied to the thin film surface after partial curing of the first (bottom) layer, affixing itself to this bottom layer. Said dry coat layer and similar layers are especially effective with low dose active ingredients that require a very low moisture environment to remain stable. The second layer can also contain substrates and partitioning agents.

The film is of a size such that it is fast dissolving. The weight per strip may vary. Said weight of the strip may be in the ranges of about 10 to 80 mg, about 20 to 70 mg, about 30 to 60 mg and about 50 mg. The maximum dosing per strip may also vary depending on the choice of active ingredient. Said maximum dosing is preferably 12.5 mg. Active ingredients can be delivered in a solid or liquid format and depending on dose levels, the Active ingredients can be oil or water soluble. Active ingredients that are stable in aqueous systems are preferred. Active ingredients that are not stable in an aqueous system, however, though not preferred, may still be used. Preferably, the dosage per serving is 1-2 strips but may vary depending on the size of the individual strip and other factors known one skilled in the art.

Individual strips can be made in virtually any size, preferably the strips are 13/16 inch by 1¼ inch rectangles. The thickness of the first layer is preferably in a range between about 0.040 to 1.1 micrometers. The thickness of the second dry coat layer is preferably in the range of about 0.007 to 0.02 micrometers. The thickness of the particularly layers may be more or less than the values recited herein depending on factors known to one skilled in the art such as load and processing challenges.

Any standard manufacturing procedure known in the art may be used to manufacture the film. An example of such a process can be found in U.S. Pat. No. 5,948,430 to ZERBE et al.

Further to the production method described in U.S. Pat. No. 5,948,430 to ZERBE et al., the production of an edible film according to the present invention can also include an aeration step. This step includes aerating the mass prior to application onto a substrate. Aeration is most preferably achieved through mechanical agitation, mechanical reaction, or carbon dioxide aeration. The aeration step produces an edible film having greater thickness and lower density than without aeration.

A further embodiment of the present invention includes an improved film and method for making the same. The film can be used on living cells. Formation of the medicant-containing layer in the film does not require a solvent and minimizes the likelihood of damage from heat and shear. The rate of dissolution or delivery of the medicant by the film can be readily adjusted. The medicant-containing layer, while minimizing the likelihood of heat induced medicant damage, permits heat to be utilized to form a coating on the edible film. Hydrophilic components can be readily incorporated in larger concentrations during production of the medicant-containing layer.

Further, the present invention includes an improved composition for delivering a medicant in the oral cavity. The composition includes an applied coating and a film layer.

The film layer is made from any polymer, softener, filler, matrix, or other composition. The film has an acceptable dissolution rate in the oral cavity for a particular thickness of film. For example, if the film has a thickness of 50 microns, it may be desirable for the film to dissolve in the oral cavity within about fifteen seconds. Or it may be desirable for the film to dissolve more slowly. By way of example, and not limitation, the film can be made with pullulan, modified starch, pectin, carageenan, a maltrodextrin, or alginate.

The applied coating is a powder matrix including one or more medicants. The medicant can be contained in a powder carrier, or can itself be a powder. One advantage of the powder matrix is that it ordinarily does not require the use of a solvent. Another advantage of the powder matrix is that it ordinarily can, if desired, include in addition to the medicant a variety of different auxiliary compositions. A further advantage of the powder matrix is that it can be admixed in a fluidized bed that minimizes the generation of shear and heat. In a fluidized bed dry air or another gas is dispersed upwardly through a plurality of openings to suspend and intermix particulate. Any desired means can be used to admix powders. Another advantage of mixing or suspending powder in a fluidized bed is that the dry air suspending the powder particles tends to prevent agglomeration of the particles. The admixed powder matrix can also be stored (i.e., suspended) in the fluidized bed, prior to the application of the admixed powder matrix to the film layer. The powder matrix can be applied in any desired manner, including sifting, screening, atomization, static, mechanical agitation, etc. For example, the powder matrix can be atomized through a Nordson or similar static spray gun using compressed air. One such gun creates a fine mist spray of powder particles. The gun statically electrically charges the powder particles so they adhere to a surface of the film layer that is receiving the powder particles. Another process for applying the powder particles is to admix the particles with a liquid carrier to form a particle—liquid solution. The particle—liquid solution is sprayed on the film layer. The liquid carrier evaporates, leaving the powder particles on the film. The liquid carrier preferably does not cause the powder particles to dissolve in the liquid carrier.

One auxiliary composition that can be included in the powder matrix with the medicant is a composition that dissolves slowly over a selected period of time. Such an auxiliary dissolution control composition can be utilized to slow the release of medicant in the oral cavity. Examples of this kind of auxiliary composition are, without limitation, gel forming compositions like carrageenan, gelatin, alignates, pullulan, PVP, and other hydrophilic materials; cyclodextrin; and, inert materials like calcium and fibers. For example, the fibers can comprise carboxymethylcellulose.

Another auxiliary composition the can be included in the powder matrix with the medicant is an absorption composition that absorbs water or saliva. Such an auxiliary absorption composition can be also be used to slow the release of medicant, and/or, to form a gel. The gel can, if desired, cause the strip to become chewable, similar to a very soft jelly-bean. As used herein, an auxiliary composition is termed a gel if, when it is placed in the oral cavity or in contact with another source of bodily liquid, (1) the auxiliary composition absorbs at least four times it weight of water or of saliva or other aqueous solution in a selected period of time, or (2) the auxiliary composition swells to at least three times its thickness in a selected period of time. The selected period of time can vary but preferably is from five seconds to fifteen minutes, most preferably five seconds to five minutes. Examples of gel auxiliary compositions include, without limitation, carboxymethylcellulose, pectin, modified starches, gelatin, and carrageenan. These compositions can be used alone or in combination. One advantage of a gel is that it tends to slow the dissolution of the medicant and to maintain the medicant in the oral cavity for a longer period of time.

A further auxiliary composition that can be included in the powder matrix is a composition that, when placed in the oral cavity in contact with the mucosa therein, adheres to the mucosa. The concentration of such auxiliary adhesion compositions in the powder matrix can be adjusted to vary the length of time that the film adheres to the mucosa or to vary the adhesive forces generated between the film and mucosa. The auxiliary adhesion compositions adhere to the oral mucosa or to mucosa or tissue in other parts of the body, including the mouth, nose, eyes, vagina, and rectum. Examples of auxiliary adhesion compositions include carboxymethycellulose, polyvinyl alcohol, polyvinyl pyrrolidone (povidone), sodiumalginate, methyl cellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl cetate, dimenthylpolysiloxanes, polyoxyalkylene block copolymers, and hydroxyethylmethacrylate copolymers. All examples of composition provided herein are given without limiting the use or inclusion of other comparable or functionally equivalent compositions even though such comparable or functionally equivalent compositions are not listed.

Still another auxiliary composition that can be included in the powder matrix is a flow composition that, when subjected to a curing process, flows to form a smoother or shinier coating on the exterior of the film layer. One preferred curing process is heating the film layer with powder coating to a selected temperature above 76 degrees F. to cause the auxiliary flow composition to soften and flow. Examples of this kind of auxiliary composition are lipids (including various animal and vegetable fats) waxes, particularly low melting point waxes, and polyols, particularly low melting point polyols that can be admixed in powder form or than can included be in powder particles containing a medicant or other compositions. The medicant itself, may also have the property of flowing at an elevated temperature in excess of 76 degrees F. to form a smoother or shinier coating.

Other auxiliary compositions that can be included in the powder matrix include, without limitation, bulking agents, fillers, pigments (coloring), flavorings, and sweeteners.

Combinations of auxiliary compositions can be included in the powder matrix to achieve a desired function. For example, if it is desired to slow the dissolution of a medicant, less soluble fillers and fibers can be included in the powder matrix along with a high concentration of polymers that have a very high degree of ability to adhere to the oral mucosa lining the mouth.

The powder matrix is normally administered to the film layer to form the applied coating after the film layer has been manufactured.

The dry powder matrix will normally contain a minor amount of retained or bound water or other liquid, typically less than about ten percent by weight. The level of moisture in the powder matrix normally should not cause the powder particles to stick or adhere to one another during intermixing of powders to form the powder matrix and during application of the powder matrix to the film layer.

By way of example, and not limitation, the film layer can be produced using a highly water-soluble polymer comprising a natural or synthetic water-soluble polymer. The polymer preferably has good film moldability, produces a soft flexible film, and is safe for human consumption. One such polymer can be a water-soluble cellulose derivative like hydroxypropyl cellulose (HPC), methyl cellulose, hydroxypropyl alkylcellulose, carboxymethyl cellulose or the salt of carboxymethyl cellulose. Or, the polymer can comprise an acrylic acid copolymer or its sodium, potassium or ammonium salt. The acrylic acid copolymer or its salt can be combined with methacrylic acid, styrene or vinyl type of ether as a comonomer, poly vinyl alcohol, poly vinyl pyrrolidone, polyalkylene blycol, hydroxy propyl starch, alginic acid or its salt, poly-saccharide or its derivatives such as trangacanth, bum gelatin, collagen, denatured gelatin, and collagen treated with succinic acid or anhydrous phthalic acid. By way of example, the following can be included in the powder matrix as adhesives: poorly water-soluble cellulose derivatives including ethyl cellulose, cellulose acetate and butyl cellulose; shellac; higher fatty acids including steric acid and palmitic acid. The following can also, without limitation, be used to produce the film layer: pullulan, maltodextrin, pectin, alginates, carrageenan, guar gum, other gelatins, etc.

Bulking agents that can be included in the powder matrix include, by way of example and not limitation, avicel, sugar alchohols including manitol and sorbitol and xylitol and isomalt, lactic sugar, sorbitol dextrin, starch, anhydrous calcium phosphate, calcium carbonate, magnesium trisilicate, silica, and amylase.

The size of particulate in the powder matrix can vary as desired, but is preferably in the range of 10 mesh to 400 mesh or finer, preferably 40 mesh to 300 mesh.

The thickness of the film layer can vary as desired, but typically is in the range of 0.01 mm to 3.00 mm, preferably 0.03 mm to 1.00 mm.

The powder matrix can be applied to one or both sides of the film layer. The film layer includes upper outer surface on the top of the film layer and includes a lower outer surface on the bottom of the film. The upper outer surface is generally parallel to the lower outer surface. The top of the film is generally parallel to the bottom of the film. The thickness of the powder matrix layer can vary as desired, but is preferably in the range of 0.001 mm to 3.00 mm, preferably 0.01 mm to 1.00 mm.

If desired, after the powder matrix layer is applied to the film layer, an additional layer or layers can be applied over the powder matrix layer to seal the powder matrix layer, slow the dissolution of the medicant from the powder matrix layer, etc.

If desired, multiple powder matrix layers can be applied to the film layer. The film layer can comprise a laminate of two or more layers. Methods for producing the film layer and incorporating plasticizers, bulking agents, taste modifying agents, pigments, etc. in the film layer are well known in the art and not described in detail herein. Since the medicant is being applied to the film layer in a dry powder form, the likelihood of adverse interactions between the medicant and compositions comprising the film layer is lessened.

Unless otherwise specified or required by the context, the term edible as used herein is used interchangeably with the term orally consumable, and generally means that the article may be placed in the mouth, oral cavity, on the tongue, or the like, without significant detrimental effect to the recipient.

In certain embodiments the compositions and films of the present invention may contain at least one flavoring and/or odorant composition that renders the composition or film palatable. Any effective flavor or odor may be used. The flavoring or odor agent or agents are present in any effective amount, including, for example, in an amount ranging from about 0.5 to 40 wt. %, 1 to 30 wt. %, 5 to 15 wt. %, 0.5 to 15 wt. %. The flavorings may be natural or artificial, or combinations thereof.

Unless otherwise specified or required by the context, the edible films of the present invention may be manufactured in any effective manner. U.S. Patent Application Nos. 20010022964, 20020131990 and 20020019447 and U.S. Pat. Nos. 6,419,903, 3,931,146, 5,411,945, 6,010,716, 5,629,003, 5,948,430, 6,177,096, 6,284,264, 5,700,478, 6,449,925, 4,072,551, 4,083,741, all of which are incorporated herein by reference as if fully set forth herein, describe methods for making edible films. These, and other methods known in the art, or described herein, may be used in accordance with the present invention.

### EXAMPLES

### Example Application 1

### Nutrient Deficiency Through Excessive Fluid Loss

Nutrient loss thorough diarrhea and vomiting in particular can cause a severe condition, especially in infants and young children, and may result in death. Diarrhea frequently involves colonization of the small intestine with enteropathogenic strains of E. Coli which produce heat stable and/or heat labile enterotoxins. Related enterotoxins are produced by other enteropthogens such as cholera, and also cause diarrhea. These enterotoxins stimulate fluid secretion in the gut lumen and cause diarrhea. Associated fluid loss may lead to death.

In cases of severe dehydration corrective parenteral (intravenous) therapy is often necessary. In cases of mild to moderate dehydration, oral rehydration solutions provide a safe and economical alternative to intravenous therapy. Oral electrolyte solutions used in oral maintenance or rehydration therapy consist of a mixture of electrolytes and a carbohydrate component such as glucose or dextrose.

Transmucosal Nutritional Supplements may be used in mild, moderate, and/or sewer cases of nutrient loss through diarrhea and vomiting.

### Example Application 2

### Nutrient Deficiency in the Aging Population

Between 50% and 75% of America's 3 million nursing borne residents have some difficulty in swallowing. In one Canadian nursing home study of 349 patients, 68% exhibited signs of dysphagia, and 40% exhibited challenging behaviors when asked to swallow medication.

Dosing these millions of patients presents a great challenge to nursing homes, menial institutions, and even general hospitals.

A study by the Department of Health & Human Services reported to Congress on Feb. 17, 2002 found that more than 90% of nursing homes are undenauffed, and would have to spend an unbudgeted $7.8 billion a year to meet even marginal care standards. Patients with chronic diseases suffer the most because of inadequate medication-administration support.

In both home care and nursing homes, the psychiatric effects of aging frequently complicate administration of solid dosage forms. Prevalence of agitation in the nursing home environment ranges from 75% to 90%; incidence of psychosis in patients with Alzheiiner's disease is 20% at the one-year benchmark and 50% at three years. The hassle of attempting to medicate these patients at home with oral solid dosage forms leads to caregiver burnout, and eventually, institutionalization of the patient. Typical of the disorders that involve underappreciated swallowing difficulties.

Parkinson's disease: One of the mast visible symptoms of Parkinson's disease is drooling, which affects 80% to 90% of patients. Drooling results from an inability to swallow saliva, not overproduction—in fact, Parkinson's disease patients actually produce less saliva than normal people. Anticholinergic drugs commonly prescribed to “dry up” excess saliva actually results in sticky saliva that is even more difficult to swallow.

Other patients with chronic swallowing difficulties include those with chronic obstructive lung disease, stroke, and Alzheimer's disease, and those with diseases and radiation therapy to the head and neck.

Still another reason for considering transmucosal drug delivery may be the reduction or elimination of hepatic metabolism. The liver significantly alters some drugs, like hormones. For others, first-pass hepatic metabolism may impair the metabolism of other drugs with which the patient is being treated.

Early testing of oral drugs in healthy volunteers can mask difficulties in dosage once actual patients are being treated. It is one thing to produce pharmacokinetic curves for an oral 5-HT3 antagonist in normal volunteers—it may be quite another to use those curves to predict how much drug was absorbed by a patient who vomited due to chemotherapy, shortly after taking a capsule.

Finally, some medications, when delivered as oral solid dosage firms, expose the entire body or specific organs to unacceptable drug levels, at least for some patients. Examples of those drugs include the NSAIDs, erectile-dysfunction treatments, and antifungals.

In the case of NSAIDs, systemic administration of oral solid dosage forms, usually to treat a highly localized pain, results in blood levels that induce GI bleeding responsible for approximately 76,000 hospitalizations and 7,600 deaths annually.

Accordingly, Transmucosal Nutritional Supplements may be of benefit in cases such as the elderly, infants, and other situations in which oral delivery may not be the preferred option.

### Example Application 3

### Nutrient Deficiency and Hyponatremia (Low Blood Sodium)

In September 1999, a 19-year-old U.S. Air Force recruit collapsed during a 5.8-mile walk, with a body temperature of 108 degrees Fahrenheit. Doctors concluded he had died of both heat stroke and low blood sodium levels as a result of overhydration.

During January 2000, a 20-year-old trainee in the U.S. Army drank around 12 quarts of water during a 2- to 4-hour period while trying to produce a urine specimen for a drug test. She then experienced fecal incontinence, lost consciousness and became confused, then died from swelling in the brain and lungs as a result of low blood sodium.

In March 2001, a 19-year-old U Marine died from drinking too much water after a 26-mile march, during which he carried a pack and gear weighing mare than 90 pounds. Although he appeared fine during the beginning stages of the 8-hour walk, towards the end he began vomiting and appeared overly tired. He was then sent to the hospital, where he fell into a coma, developed brain swelling and died the next day. It is unclear how much water he drank during the march, but Marines were given a “constant emphasis” on drinking water before and during the activity, Gardner writes in the latest issue of Military Medicine.

Accordingly, Transmucosal Nutritional Supplements (sodium supplements) may be of benefit in preventing Hypoatremia.

### Example Application 4

### Iron Deficiency

Anemia is a condition where red blood cells are not providing adequate oxygen to body tissues. There are many types and causes anemia. Iron deficiency anemia is a decrease in the number of red cells in the blood caused by too little iron.

Iron deficiency anemia is the most common form of anemia. Approximately 20% of women, 50% of pregnant women, and 3% of men are iron deficient. Iron is an essential component of hemoglobin, the oxygen-carrying pigment in the blood. Iron is normally obtained though the food in the diet and by recycling iron from old red blood cells. Without it, the blood cannot carry oxygen effectively and oxygen is needed for the normal functioning of every cell in the body.

The causes of iron deficiency are too little iron in the diet, poor absorption of iron by the body, and loss of blood (including from heavy menstrual bleeding). It can also be related to lead poisoning in children.

Anemia develops slowly after the normal stores of iron have been depleted in the body and in the bone marrow. Women, in general, have smaller stores of iron than men and have increased loss through menstruation, placing them at higher risk for anemia than men.

In men and postmenopausal women, anemia is usually caused by gastrointestinal blood loss associated with ulcers the use of aspirin or nonsteroidal anti-inflammatory medications (NSAJDS), or certain types of cancer (esophagus, stomach, colon).

High-risk groups include women of child-bearing age who have blood loss through menstruation; pregnant or lactating women who have an increased requirement for iron; infants, children, and adolescents in rapid growth phases, and people with a poor dietary intake of iron. Risk factors related to blood loss are peptic ulcer disease, long term aspirin use and colon cancer.

The cause of the deficiency must be identified, particularly in older patients who are most susceptible to intestinal cancer.

Oral iron supplements are available (ferrous sulfate). The best absorption of iron is on an empty stomach, but many people are unable to tolerate this and may need to take it with food. Milk and antacids may interfere with absorption of iron and should not be taken at the same time as iron supplements. Vitamin C can increase absorption and is essential in the production of hemoglobin.

Supplemental iron is needed during pregnancy and lactation because normal dietary intake rarely supplies the required amount.

The hematocrit should return to normal after 2 months of iron therapy, but the iron should be continued for another 6 to 12 months to replenish the body's iron stores, which are contained mostly in the bone marrow.

Intravenous or intra-muscular iron is available for patients who can't tolerate oral forms.

Iron-rich foods include raisins, meats (liver is the highest source), fish, poultry, eggs (yolk), legumes (peas and beans), and whole grain bread.

Accordingly, Transmucosal Nutritional Supplements (iron supplements) may be of benefit as a source of iron, and in the prevention of iron deficiency/anemia

### Example Application 5

### Calcium Deficiency

Calcium is essential for many body functions, including regulation of the heartbeat, conduction of nerve impulses, stimulation of hormone secretions and clotting of blood, as well as for building and maintaining a healthy skeleton.

Calcium is a mineral found in many foods and adequate calcium intake is important because the human body cannot produce calcium. Even after reaching full skeletal growth, adequate calcium intake is important because the body loses calcium every day through shed mucous membrane, nails, hair, and sweat as well as through urine and frees. This lost calcium must be replaced daily through the diet. When the diet does not contain enough calcium to perform these activities, calcium is taken from the bones, the storage area for calcium.

The National Academy of Sciences and the National Osteoporosis Foundation recommend daily calcium intakes of 1000-1200 mg/day for adult men and women. According to experts, food is the best source of calcium; however, most Americans do riot have enough calcium in their diets. Fortunately, calcium-fortified foods and calcium supplements can fill the gap, ensuring that the daily calcium requirement is met. The amount needed from a supplement depends on how much calcium is consumed from food sources.

Calcium exists in nature only in combination with other substances called compounds. Several different calcium compounds are used in supplements including calcium carbonate, calcium phosphate and calcium citrate. These compounds contain different amounts of elemental calcium, which is the actual amount of calcium in the supplement. It is important to read the label carefully to determine how much elemental calcium is in the supplement and how many doses or pills to take.

Calcium supplements are available without a prescription in a wide range of preparations and strengths, which can make selecting one a confusing experience. Many people ask which calcium supplement they should take; the “best” supplement is the one that meets an individual's needs based on tolerance, convenience, cost and availability. In choosing a calcium supplement, the following are important considerations:

Purity—Choose calcium supplements that are known brand names with proven reliability. Look for labels that state “purified” or have the USP (United States Pharmacopeia) symbol. Since applying for the USP symbol is voluntary, however, many fine products may not display this symbol. Avoid calcium from unrefined oyster shell, bone meal or dolomite without the USP, as these historically have contained higher lead levels or other toxic metals.

Absorbability—Most brand name calcium products are absorbed easily in the body. If the product information does not state that it is absorbable, how well a tablet dissolves can be determined by placing it in a small a of warm water for 30 minutes, stirring it occasionally. If hasn't dissolved within this time it probably will not dissolve in the stomach. Chewable and liquid calcium supplements dissolve well because they are broken down before they enter the stomach. Calcium, whether from the diet or supplements, is absorbed best by the body when it is taken several times a day in amounts of 500 mg or less, but taking it all at once is better than not taking it at all. Calcium carbonate is absorbed best when taken with food. Calcium citrate can be taken anytime.

Tolerance—While calcium supplements generally are a satisfactory option for many people, certain preparations may cause side effects, such as gas or constipation, in some individuals. If simple measures such as increased fluids and fiber intake do not solve the problem, another form of calcium should be tried. Also, it is important to increase supplement intake gradually; take 500 mg a day for a week, then add more calcium slowly.

Calcium Interactions—It is important to talk with a physician or pharmacist about possible interactions between prescription or over-the-counter medications and calcium supplements. For example, calcium supplements also may reduce the absorption of the antibiotic tetracycline. Calcium also interferes with iron absorption, so a calcium supplement should not be taken at the same time as an iron supplement. The exception to this is when the iron supplement is taken with vitamin C or calcium citrate. Any medication to be taken on an empty stomach should not be taken with calcium supplements.

Combination Products—Calcium supplements are available in a dazzling array of combinations with vitamins and other minerals. While vitamin D is necessary for the absorption of calcium, it is not necessary that it be in the calcium supplement (see winter 1998 issue of Osteoporosis Report for information on vitamin D). Minerals such as magnesium and phosphorus also are important, but usually are obtained through food or multivitamins. Most experts recommend that nutrients come from a balanced diet, with multivitamins used to supplement dietary deficiencies.

Most published studies show that low calcium intake is associated with low bone mass, rapid bone loss and high fracture rates. Adequate calcium intake will help ensure that calcium deficiency is not contributing to a weakening of the skeleton; however, this is only one of the steps necessary for bone health. A high calcium intake will not protect a person against bone loss caused by estrogen deficiency, physical inactivity, smoking, alcohol abuse or various medical disorders or treatments.

Accordingly, Transmucosal Nutritional Supplements (calcium supplements) may be of benefit as a source of calcium, and in the prevention of calcium deficiency.

### Example Application 6

### Micronutrient Influence Upon Muscle Contractions (Muscle Cramps)

Muscle cramps are sudden electrically active contractions elicited by motor neuron hyperexcitement, or the inability of the myosin head to release from its attraction to the actin head protein. Some have assumed that exertional cramping may be the result of fluid electrolyte improprieties. Modem research science is divided on the importance of sweat losses of sodium, chloride, potassium, and magnesium and regards them as trivial, therefore not evidential as a primary cause of the “Rigor Complex”. There is, however, some preliminary evidence which indicates that ATP translocation is associated with sodium, potassium, -ATP-ase. Balance of fluid ratio and electrolyte intracellular to extracellular levels in the presence of Adenosine Triphosphate and its Adenosine Triphosphatase enzyme would appear mandatory for optimum muscle function. A nationally ranked tennis player has experienced unexplainable muscle heat cramps during play. Medical examinations and history were unremarkable, and were confirmed by in-patient blood serum profiles. On court evaluation of sweat loss composition and a 3-day dietary analysis revealed that sodium loss during play far exceeded dietary intake. Increase of daily dietary sodium chloride eliminated heat cramps reoccurrence.

Among the elderly, frequent cramping caused by compromised circulation may provide a model for the extreme, but similar physiological environment experience by an athlete during heat stress. Idiopathic cramping among older people was found to be directly related to electrolyte deficiencies, heat stress, metabolic myopathies, thyroid disease, dystonias, reaction to medications, and hemodialysis. It is suggested that treatment include stretching, oral Vitamin E, and/or Quinine Sulfate supplements. Further, no single treatment results in one effective remedy.

Dr. T. D. Noakes (1991) summarizes exertional cramps as follows: (1) Exhaustion related to glycogen depletion for fresh ATP replenishment, (2) Excessive fluid volume to electrolyte profile. He suggests intake of 16 ounces fluid using 60-120 grams carbohydrates prior to and during each how of prolonged endurance training.

CALCIUM: IONIZED MINERAL OF INTEREST—During extraordinary muscle energy metabolism, mineral flux may deplete or vary normal electrolyte homeostatic ratios. Calcium is the most abundant mineral and the fifth most abundant element found in the human body. It is therefore vital to muscle contraction, nerve transmission, blood clotting, and a multiple of metabolic functions. Bones act as a calcium reservoir, providing it when blood serum values decline below 10 mg/100 ml, regulated by parathyroid hormonal controls. Over half of the serum calcium is ionized, while the remainder is protein-bound or associated with organic and inorganic acids. Protein-bound calcium acts as a weak electrolyte, while metabolically active ionized calcium is used by the blood and serum for muscle contraction.

During exercise blood calcium falls, arousing the parathyroid gland to stimulate vitamin D activation of ionized calcium release from bone stores. As calcium levels are reinstated, parathyroid stimulation halts, and calcitonin from the thyroid is released, thereby halting bone resorption/release. (Garrison & Somer 1995) Calcium depletion sensitizes neural muscle tetany. Calcium is vital to synaptic release of neurotransmitter substances which enable nerves to excite and relax during muscle contractions. The volume of neurotransmitter release is proportionate to ionized calcium concentration in the terminal membrane, and inversely proportionate to magnesium concentration. Serotonin, Acetylcholine, and Norephinephrine transmitter levels are affected by the enzymatic influences of both calcium and magnesium upon striated and smooth muscle contraction. Without substantial amounts of calcium, the glycogen enzyme, phosphorylase kinase is not able to breakdown glycogen to glucose-6-P for energy metabolisms. Calcium also activates the adenosine triphosphatase enzyme for the hydrolysis of ATP. Dr. Balch (1990) stated that muscle cramps are “Commonly caused by a calcium-magnesium imbalance and/or vitamin E deficiency.” He recommends a daily dietary or supplemental intake of 2:1 Calcium (1500 mg.) to Magnesium (750 mg.) and 400-1000 IU of vitamin E for prevention of muscle cramps. Substantial research noted leg cramps during pregnancy were caused by alterations of calcium metabolism. (Pitikin 1983) Studies by Hammar (1987), Knowles (1981), Odendahl (1974), and Page (1953) further suggest that supplementation of calcium or reduction of phosphorus may prevent and relieve such cramping in the legs. Possibly related to calcium balance, reduced serum magnesium has been associated with tetany and muscle cramping. Similar findings have confirmed evidence when supplemental ingestion of calcium and magnesium relieved tetanical symptoms.

ELECTROLYTE BALANCE: DELICATE AND DELIBERATE—The Cations and Anions of fluid electrolyte composition are never static, but are proportionately balanced within the compensatory rates of metobalic activity both intracellularly and extracellularly. Pivotal losses of calcium and magnesium from muscle exhaustion, fluid dehydration from sweat loss, depletion of extracellular cation stores of sodium or intracellular cation stores of potassium are significant factors staged for muscle failure, i.e., a cramp event. While the previously mentioned case study of a nationally-ranked tennis player whose severe exertional muscle cramps were solved by dietary sodium supplementation, modern science considers one solution insignificant in terms of scientific methodology for settling on conclusive evidence. Muscle cramps have been associated ‘with a hypokalemic tissue environment, and were readily relieved by potassium supplements. (Portier 1973) Glatzel (1980) was successfully treating nocturnal cramps with dietary sodium chloride. Strong evidence exists for the role of electrolyte depletion associated with muscle spasms, cramps, and seizures, but inconclusive from present research literature. In fact, depletion of muscle glycogen, fluid overhydration, and the lack of vitamin substrates with enzymatic influence en fuel selection are also presently considered suspects.

VITAMINS NECESSARY FOR FUEL CONVERSION AND FREE RADICAL SCAVENGING: VITAMIN 8-6, VITAMIN E—Vitamin E supplementation to shown to relieve muscle tramping in several clinical observations by Lotzof (1977) and Cathcart (1972). Two separate experimental studies by Ayres (1969 & 1974) confirmed the findings of Catheart amd. Lotzof. Dr. Balch's research of the literature (1990) recently was added to the aforementioned scholarship. Nocturnal muscle spasms and distal/peripheral small muscle cramps were releived by oral ingestion of vitamin B-6 (Pyrodoxine) in the studies performed by Ellis and Presley (1973)

Accordingly, Transmucosal Nutritional Supplements may be of benefit in alleviating or extending the onset of muscle contractions.

### Example Application 7

### Oral/Buccal Transmucosal Nutrient Delivery as an Athletic Supplement to Combat Nutrients Lost Through Excessive Fluid Loss

Another aspect of the present invention relates to transmucosal methods and products for replenishing nutrients and supplying additional components to a subject involved in strenuous exercise, including eletrolytes, which avoids many of the limitations associated with sports drinks or other orally ingested nutritional supplements.

The invention in one aspect is a quick-dissolve strip (or reservoir pouch/packet) for administering nutrients utilized during exercise and other periods of high energy exertion.

The nutrients are delivered from the quick strip directly to the blood stream where they can supply the necessary energy or maintenance of homeostatic conditions in the body. There is no need for the nutrients to pass through the gastrointestinal tract where absorption would be a limiting factor.

The delivery of an athletic supplement using a quick-dissolve strip offers several advantages over traditional delivery methods. For instance, the quick-dissolve strip avoids gastrointestinal metabolism of the athletic supplement, reduces first pass effects and may if desirable provide a longer course of release of the components of the athletic supplement than traditional methods such as the use of sports drinks or other orally ingested nutritional supplements.

The quick-dissolve strip may include many different concentrations of the components of the athletic supplement.

The quick-dissolve strip may be any type of conventional quick-dissolve strip, such as a transmucosal quick-dissolve strip, a sublingual quick-dissolve strip, or a buccal quick-dissolve strip. In a preferred embodiment the quick-dissolve strip includes a permeation enhancing amount of at least one mucous membrane permeation enhancer.

The transmucal quickdissolve strip may be of any shape, such as oblong, square, round, rectangular, etc. The transmucosal quick-dissolve strip may also have a variety of sizes.

The quick-dissolve strip of the present embodiment provides all of the nutritional, carbohydrate, and energy requirements of an athlete under conditions of physical stress without causing gastrointestinal disturbances.

Accordibngly, Transmucosal Nutritional Supplements (electrolyte/other supplements) may be of benefit to replace nutrients lost through excessive fluid loss from physical activities.

### Example Appication 8

### Tranmucosal Nutrient Product Mix (Composition)

The following is a formulation for a Transmucosal Nutrient Supplement composition (for excessive body fluid loss)

1 part Potassium Chloride

1.93 parts Trisodium Citrate

2.33 parts Sodium Chloride

13.33 parts Glucose

Said formualtion is to be combined with proper fluid (water) rehydration guidelines.

### Example Application 9

### Vitamin Deficiencies, Micronutrient Deficiencies

Dietary deficiencies of vitamins and minerals—life-sustaining nutrients needed only in small quantities (hence. “micronutrients”)—cause learning disabilities, mental retardation, poor health, tow work capacity, blindness, and premature death. The result is a devastating public health problem: about 1 billion people, almost all in developing countries, are suffering the effects of these dietary deficiencies, and another billion are at risk of falling prey to them.

To grasp the enormous implications at the country level, consider a country of 50 million people with the levels of micronutrient deficiencies that exist today in South Asia. Such a country would suffer the following losses each year because of these deficiencies:

20,000 deaths

11,000 children born cretins or blinded as preschoolers

1.3 million person-years of work lost due to lethargy or more severe disability

360,000 student-years wasted (3 percent of total student body)

In terms of losses by type of deficiency, more than 13 million people suffer night blindness or total blindness from the lac of vitamin A. In areas without adequate iodine in the diet, five to ten offspring of every 1,000 pregnant women are dead upon birth or soon thereafter due to iodine deficiency. Severe iron deficiency causes as many as one in five maternal deaths, as well as the death of about 30 percent of children who enter the hospital with it and do not get a blood trransfusion (those who do get the transfusion are exposed to other risks).

Accordingly, Transmucosal Nutritional Supplements (micronutrient supplements) may be of benefit for vitamin/micronutrient deficiencies worldwide.

While the invention is described in terms of a specific embodiment, other embodiments could readily be adapted by one skilled in the art. Accordingly, the scope of the present invention is limited only by the following claims.

1. A composition comprising:
a film layer;
a coating, wherein the coating is applied to at least one side of the film layer; and
a nutritional supplement.

2. The composition of claim 1 wherein the coating comprises a powder matrix.

3. The composition of claim 1 wherein the film layer comprises the nutritional supplement.

4. The composition of claim 2 wherein the powder matrix comprises the nutritional supplement.

5. The composition of claim 4 wherein the nutritional supplement is selected from the group consisting of Iron, Sodium, Calcium, Magnesium, Carbohydrates, Proteins, Zinc, Molybdenum, Copper, Potassium, Manganese, Chlorides, Bicarbonate and Carbonate, Aluminium, Arsenic, Bromine, Cadmium, Chromium, Chlorine, Cobalt, Fluorine, Iodine, Manganese, Molybdenum Nickel, Phosphorus, Selenium, Silicon, Vanadium, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantethine, Folic Acid, Vitamin B12, “Unofficial” B Vitamins including Choline and Inositol, Vitamin P (bioflavonoids).

6. The composition of claim 4 wherein the nutritional supplement comprises an electrolyte.

7. The composition of claim 4 wherein the nutritional supplement comprises a vitamin.

8. The composition of claim 6 wherein the powder matrix comprises an auxiliary dissolution control composition.

9. The composition of claim 8 wherein the auxiliary dissolution control composition comprises one or more of carrageenan, gelatin alginates, pullulan, PVP, cyclodextrin, calcium, or fibers.

10. The composition of claim 6 wherein the powder matrix comprises an absorption composition.

11. The composition of claim 10 wherein the absorption composition comprises one or more of carboxymethylcellulose, pectin, modified starches, gelatin, or carrageenan.

12. The composition of claim 6 wherein the powder matrix comprises an adhesive.

13. The composition of claim 12 wherein the adhesive comprises one or more of poorly water soluble cellulose derivatives including ethyl cellulose, cellulose acetate and butyl cellulose, shellac, or fatty acids including steric acid and palmitic acid.

14. The composition of claim 6 wherein the powder matrix further comprises a mucosa adherent.

15. The composition of claim 14 wherein the mucosal adherent is selected from one or more of carboxymethylcellulose, polyvinyl alcohol, polyvinyl pyrrolidone, sodiumalginate, methylcellulose, hydroxyl propyl cellulose, hydroxypropylmethyl cellulose, polyethylene glycols, carbopol, polycarbophil, carboxyvinyl copolymers, propylene glycol alginate, alginic acid, methyl methacrylate copolymers, tragacanth gum, guar gum, karaya gum, ethylene vinyl cetate, dimenthylpolysiloxanes, polyoxyalkylene block copolymers or hydroxyethylmethacrylate copolymers.

16. The composition of claim 6 wherein the powder matrix comprises a flow agent.

17. The composition of claim 16 wherein the flow agent is a lipid, wax or polyol.

18. The composition of claim 6 wherein the powder matrix comprises a bulking agent.

19. The composition of claim 19 wherein the bulking agent comprises one or more of avicel, sugar alcohols including manitol, sorbitol, xylitol and isomalt, lactic sugar, sorbitol dextrin, starch, anhydrous calcium phosphate, calcium carbonate, magnesium trisilicate, silica or amylase.

20. The composition of claim 6 wherein the powder matrix further comprises one or more of a bulking agent, filler, pigment, flavoring agent, or sweetener.

21. The composition of claim 6 wherein the powder matrix comprises less than about 10% water by weight.

22. The composition of claim 6 wherein the thickness of the film layer is in the range of about 0.01 mm to about 3 mm.

23. The composition of claim 6 wherein the thickness of the film layer is in the range of about 0.03 mm to about 1 mm.

24. The composition of claim 6 wherein the film layer comprises at least two layers.

25. A composition comprising:
a film layer wherein the film layer rapidly dissolves in an oral cavity; and
a powder coating comprising a nutritional supplement wherein the powder coating is applied to at least one side of the film layer.

26. The composition of claim 25 wherein the film layer dissolves within thirty seconds of being placed in the oral cavity.

27. The composition of claim 25 wherein the film layer dissolves within 15 seconds of being placed in the oral cavity.

28. The composition of claim 25 wherein the nutritional supplement is selected from the group consisting of Iron, Sodium, Calcium, Magnesium, Carbohydrates, Proteins, Molybdenum, Copper, Potassium, Manganese, Chlorides, Bicarbonate and Carbonate, Aluminium, Arsenic, Bromine, Cadmium, Chromium, Chlorine, Cobalt, Fluorine, Iodine, Manganese, Molybdenum Nickel, Phosphorus, Selenium, Silicon, Vanadium, Zinc, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantethine, Folic Acid, Vitamin B12, “Unofficial” B Vitamins including Choline and Inositol, and Vitamin P (bioflavonoids).

29. The composition of claim 25 wherein the nutritional supplement comprises an electrolyte.

30. The composition of claim 25 wherein the nutritional supplement comprises a vitamin.

31. The composition of claim 25 wherein the film layer comprises one or more of pullulan, modified starch, pectin, carageenan, a maltrodextrin or alginate.

32. The composition of claim 25 wherein the film layer comprises a natural or synthetic water soluble polymer.

33. A composition comprising:
a film layer wherein the film layer rapidly dissolves in an oral cavity; and
a coating comprising a powder matrix;
wherein the coating is applied to at least one side of the film layer and wherein the powder matrix comprises a nutritional supplement, an adhesive, a bulking agent, a flow agent, and a sweetener.

34. The composition of claim 33 wherein the film layer dissolves within thirty seconds of being placed in the oral cavity.

35. The composition of claim 33 wherein the film layer dissolves within fifteen seconds of being placed in the oral cavity.

36. The composition of claim 33 wherein the nutritional supplement is selected from the group consisting of Iron, Sodium, Calcium, Magnesium, Carbohydrates, Proteins, Molybdenum, Copper, Potassium, Manganese, Chlorides, Bicarbonate and Carbonate, Aluminium, Arsenic, Bromine, Cadmium, Chromium, Chlorine, Cobalt, Fluorine, Iodine, Manganese, Molybdenum Nickel, Phosphorus, Selenium, Silicon, Vanadium, Zinc, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantethine, Folic Acid, Vitamin B12, “Unofficial” B Vitamins including Choline and Inositol, Vitamin P (bioflavonoids).

37. The composition of claim 33 wherein the nutritional supplement comprises an electrolyte.

38. A method of manufacturing a rapidly dissolving thin film comprising the steps of:
providing a film layer;
applying a coating to said film layer wherein the coating comprises a powder matrix and wherein the powder matrix comprises a nutritional supplement, an adhesive, a bulking agent, a flow agent, and a sweetener.

39. The method of claim 38 wherein the film layer dissolves within fifteen seconds of being placed in the oral cavity.

40. The method of claim 38 further comprising the step of drying the film layer and powder matrix.

41. The method of claim 40 wherein the step of drying is at a temperature at about the softening point of the flow agent.

42. The method of claim 38 wherein the flow agent comprises a lipid, wax or polyol.

43. The method of claim 38 wherein the nutritional supplement is selected from the group consisting of Iron, Sodium, Calcium, Magnesium, Carbohydrates, Proteins, Molybdenum, Copper, Potassium, Manganese, Chlorides, Bicarbonate and Carbonate, Aluminium, Arsenic, Bromine, Cadmium, Chromium, Chlorine, Cobalt, Fluorine, Iodine, Manganese, Molybdenum Nickel, Phosphorus, Selenium, Silicon, Vanadium, Zinc, Amino Acids, Vitamin A, Vitamin D, Vitamin E, Vitamin K, Vitamin C, Vitamin B complex, Thiamine (Vitamin 31), Riboflavin (Vitamin 132). Niacin (Vitamin B3), Pyridoxine (Vitamin B6), Biotin, Pantothenic Acid and Pantethine, Folic Acid, Vitamin B12, “Unofficial” B Vitamins including Choline and Inositol, and Vitamin P (bioflavonoids).

44. The method of claim 38 wherein the nutritional supplement comprises an electrolyte.

45. The method of claim 38 wherein the nutritional supplement comprises a vitamin.

46. The method of claim 38 further comprising the step of preparing the coating in a fluidized bed.

47. The method of claim 38 wherein the coating is applied by sifting, screening, atomization, static or mechanical agitation.

48. The method of claim 38 wherein the powder particles are charged.

49. The method of claim 48 wherein the coating is applied using a static spray gun.

50. The method of claim 49 wherein the static spray gun charges the powder particles such that the powder particles adhere to the surface of the film layer.
